(19) United States
(12) Patent Application Publication
(10) Pub. No.: US 2022/0378590 A1
(43) Pub. Date: Dec. 1, 2022

(54) ENDOVASCULAR DEVICES, ENDOVASCULAR DEVICE SYSTEMS AND RELATED METHODS

(71) Applicant: Louis F. Knoepp, Williamston, SC (US)

(72) Inventor: Louis F. Knoepp, Williamston, SC (US)

(21) Appl. No.: 17/335,050

(22) Filed: May 31, 2021

(51) Int. Cl. A61F 2/958; A61M 25/10

(52) U.S. Cl. CPC A61F 2/958; A61M 25/10; A61M 2025/1043; A61M 2206/11

(57) ABSTRACT

Endovascular stents and endovascular device platforms and related methods that allow for the modulation/regulation of vascular flow to treat certain disease states are provided. In particular, a covered endovascular stent is provided that, upon deployment, is configured to an installed shape. A balloon catheter comprising a stem and an angioplasty balloon on an end of the stem can be provided to expand the stent. The angioplasty balloon can have a dumbbell shape when inflated with a first head on a first end of the angioplasty balloon having a first outer diameter and a second head on a second end of the angioplasty balloon having a second outer diameter and a narrow center portion having a third outer diameter that is less than the first outer diameter of the first end and the second outer diameter of the second end of the angioplasty balloon. Thereby, the covered stent configured in the installed shape can comprise a dumbbell shaped tubular structure having a first inner diameter of a first end portion and a second inner diameter of a second end portion that are larger than a third inner diameter within a center portion of the tubular structure.

### TECHNICAL FIELD

The present subject matter relates to endovascular devices and endovascular device systems and related methods that allow for the modulation/regulation of vascular flow. In particular, the present subject matter relates to endovascular stents and endovascular device systems and related methods that allow for the modulation and/or regulation of vascular flow to treat certain disease states.

### BACKGROUND

A major function of the kidneys is to remove waste products and excess fluid from the body. These waste products and excess fluid are removed through the urine. The production of urine involves highly complex steps of excretion and reabsorption. This process is necessary to maintain a stable balance of body chemicals. The critical regulation of the body's salt, potassium and acid content is performed by the kidneys. The kidneys also produce hormones that affect the function of other organs. For example, a hormone produced by the kidneys stimulates red blood cell production. Other hormones produced by the kidneys help regulate blood pressure and control calcium metabolism.

Over 650,000 Americans suffer from kidney failure. Of these, just under 500,000 require dialysis treatments. The majority of patients on dialysis undergo hemodialysis or blood related dialysis. Those patients typically have a device surgically inserted that provides access to the patient's intra-vascular blood volume. That device can be periodically connected to an artificial kidney machine. This process is called hemodialysis. While there are other types of dialysis, hemodialysis is the most common by far.

The number of patients world-wide who receive dialysis or similar measures to survive exceeds two million. Kidney failure is a lethal disease. Still, there are many who suffer from kidney-related issues that do not get the needed treatment, It is estimated that the more than two million patients who receive such dialysis or similar measures probably only account for 10% of the people worldwide that need such dialysis treatment to improve their day to day living circumstances.

Hemodialysis can require surgical devices in certain circumstances. Two classic surgically inserted devices are generally used for hemodialysis. The first type of surgical devices can comprise a venous access catheter that are usually used for short term dialysis access or as a bridge to a more long-term device. The second type of surgical device can comprise long term devices that typically revolve around creating a surgical connection between the patient's artery and vein that allows for high pressure blood to “shunt” rapidly to the low-pressure venous system. This “shunt,” or dialysis access device, allows for high flow and high-volume sampling of the patient's blood and subsequent connection to an artificial kidney machine. Once the blood is cleaned of toxins, the blood is returned to the patient via a second connection to the dialysis access device. dialysis access devices are either artificial or native to the patient (the patient's own vein). All dialysis access devices require native arterial blood (the patient's own) for correct function. While there are multiple potential complications related to dialysis access devices, arterial steal is a complication that can be detrimental to the health of the patient on dialysis.

Arterial steal or vascular access steal syndrome is a syndrome caused by ischemia (not enough blood flow to the patient) resulting from insertion of a dialysis access device. dialysis access devices function by altering the patients native vascular flow. When this flow alteration causes ischemia, it is call vascular access steal syndrome. Options for treatment of vascular access steal syndrome typically involve a surgical procedure to change the blood flow in the dialysis access device and increase the patients native flow, thus relieving the ischemia. In extreme cases, the dialysis access needs to be sacrificed or reversed, which re-directs all the blood flow back to the patient, relieving the ischemia. Such sacrificial or reversal procedures obviously lead to the “loss” of that device as an option for dialysis treatment.

As such, a need exists for a minimally invasive device and treatment for treating vascular access steal syndrome to allow patients who suffer from vascular access steal syndrome and the underlying ischemia to safely continue receiving dialysis.

### SUMMARY

The present subject matter provides endovascular stents and endovascular device platforms that allows for the modulation/regulation of vascular flow to treat certain disease states. The endovascular device platform can comprise a balloon-stent system that is inserted using minimally invasive techniques. The balloon-stent system can comprise of a dumbbell, stepped or funnel shaped angioplasty balloon with a mounted covered, vascular stent. The angioplasty balloon can be used for inserting, positioning and expanding the vascular stent in a desired vascular structure, such as a vein, artery, or artificial blood vessel. Once inserted, the stent can modulate and even reduce vascular flow. Methods related to the assembly and use of the endovascular stents and endovascular device platforms as disclosed herein are also provided.

Thus, it is an object of the presently disclosed subject matter to provide endovascular stents and endovascular device platforms as well as methods related thereto. While one or more objects of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

A full and enabling disclosure of the present subject matter including the best mode thereof to one of ordinary skill in the art is set forth more particularly in the remainder of the specification, including reference to the accompanying figures, in which:

FIG. 1A illustrates a perspective view of an embodiment of a cover stent in an installed shape according to the present subject matter;

FIG. 1B illustrates a perspective partial cross-sectional view of the embodiment of the cover stent in the installed shape according to FIG. 1A showing the contoured shape of the interior of the covered stent;

FIG. 1C illustrates a cross-sectional view of the embodiment of the cover stent in the installed shape according to FIG. 1A showing the shape of walls of the cover stent when the cover stent is in the installed shape;

FIG. 1D illustrates a schematic view of an embodiment of a cover stent in an installed shape installed in a dialysis access device within an arm of a patient according to the present subject matter;

FIG. 2A illustrates a cross-sectional view of an embodiment of a wall section of an embodiment of a cover stent in an installed shape according to the present subject matter;

FIG. 2B illustrates a cross-sectional view of another embodiment of a wall section of an embodiment of a cover stent in an installed shape according to the present subject matter;

FIG. 3 illustrates a perspective view of an embodiment of a cover stent in which the tubular structure is unexpanded in a preinstallation shape according to the present subject matter;

FIG. 4A illustrates a side plan view of an embodiment of a balloon catheter having an embodiment of an angioplasty balloon thereon in a deflated state according to the present subject matter;

FIG. 4B illustrates a side plan view of the embodiment of the balloon catheter according to FIG. 4A with the angioplasty balloon thereon in an inflated state according to the present subject matter;

FIG. 4C illustrates a side plan view of an embodiment of an endovascular stent-balloon catheter system with an embodiment of a catheter having an embodiment of an angioplasty balloon thereon in a deflated state with a covered stent mounted on the angioplasty balloon according to the present subject matter;

FIG. 4D illustrates a side plan view of the embodiment of the endovascular stent-balloon catheter system according to FIG. 4C with the angioplasty balloon thereon in an inflated state and the covered stent mounted on the angioplasty balloon expanded into an installed dumbbell shape according to the present subject matter;

FIG. 5A illustrates a schematic cross-sectional view of a vascular structure with an embodiment of an endovascular stent-balloon catheter system having an embodiment of a catheter with an embodiment of an angioplasty balloon thereon in a deflated state with a covered stent mounted on the angioplasty balloon being inserted into the vascular structure according to the present subject matter;

FIG. 5B illustrates a schematic cross-sectional view of the vascular structure according to FIG. 5A with the embodiment of the endovascular stent-balloon catheter system being positioned for installation of the covered stent within the vascular structure according to the present subject matter;

FIG. 5C illustrates a schematic cross-sectional view of the vascular structure according to FIG. 5A with the angioplasty balloon on the catheter of the embodiment of the endovascular stent-balloon catheter system being inflated to an inflated state to expand the covered stent into a installed shape within the vascular structure according to the present subject matter;

FIG. 5D illustrates a schematic cross-sectional view of the vascular structure according to FIG. 5A with the angioplasty balloon on the catheter of the embodiment of the endovascular stent-balloon catheter system being deflated and the endovascular stent-balloon catheter system being removed from the vascular structure leaving the expanded covered stent in the installed shape within the vascular structure according to the present subject matter;

FIG. 6A illustrates a schematic cross-sectional view of a vascular structure with an embodiment of a covered stent in an installed shape installed in the vascular structure with an embodiment of a balloon catheter with an embodiment of an angioplasty balloon thereon in a deflated state being maneuvered within the vascular structure for inserting the balloon catheter into the covered stent in the vascular structure according to the present subject matter;

FIG. 6B illustrates a schematic cross-sectional view of the vascular structure according to FIG. 6A with the angioplasty balloon of the balloon catheter in a deflated state being inserted into the covered stent in the vascular structure according to the present subject matter;

FIG. 6C illustrates a schematic cross-sectional view of the vascular structure according to FIG. 6A with the angioplasty balloon of the balloon catheter being inflated to expand a diameter the covered stent in the vascular structure according to the present subject matter; and

FIG. 6D illustrates a schematic cross-sectional view of the vascular structure according to FIG. 6A with the angioplasty balloon of the balloon catheter being inflated to fully expand the covered stent in the vascular structure according to the present subject matter.

Repeat use of reference characters in the present specification and drawings is intended to represent the seam or analogous features or elements of the present subject matter.

### DETAILED DESCRIPTION

Reference now will be made to the embodiments of the present subject matter, one or more examples of which are set forth below. Each example is provided by way of an explanation of the present subject matter, not as a limitation. In fact, it will be apparent to those skilled in the art that various modifications and variations can be made in the present subject matter without departing from the scope or spirit of the present subject matter. For instance, features illustrated or described as one embodiment can be used on another embodiment to yield still a further embodiment. It is to be understood by one of ordinary skill in the art that the present discussion is a description of exemplary embodiments only, and is not intended as limiting the broader aspects of the present subject matter, which broader aspects are embodied in exemplary constructions.

Although the terms first, second, right, left, front, back, top, bottom, etc. may be used herein to describe various features, elements, components, regions, layers and/or sections, these features, elements, components, regions, layers and/or sections should not be limited by these terms. These terms are only used to distinguish one feature, element, component, region, layer or section from another feature, element, component, region, layer or section. Thus, a first feature, element, component, region, layer or section discussed below could be termed a second feature, element, component, region, layer or section without departing from the teachings of the disclosure herein.

Similarly, when a feature or element is being described in the present disclosure as “on” or “over” another feature or element, it is to be understood that the features or elements can either be directly contacting each other or have another feature or element between them, unless expressly stated to the contrary. Thus, these terms are simply describing the relative position of the features or elements to each other and do not necessarily mean “on top of” since the relative position above or below depends upon the orientation of the device to the viewer.

Embodiments of the subject matter of the disclosure are described herein with reference to schematic illustrations of embodiments that may be idealized. As such, variations from the shapes and/or positions of features, elements or components within the illustrations as a result of, for example but not limited to, user preferences, manufacturing techniques and/or tolerances are expected. Shapes, sizes and/or positions of features, elements or components illustrated in the figures may also be magnified, minimized, exaggerated, shifted or simplified to facilitate explanation of the subject matter disclosed herein. Thus, the features, elements or components illustrated in the figures are schematic in nature and their shapes and/or positions are not intended to illustrate the precise configuration of the subject matter and are not necessarily intended to limit the scope of the subject matter disclosed herein unless it specifically stated otherwise herein.

It is to be understood that the ranges and limits mentioned herein include all ranges located within the prescribed limits (i.e., subranges). For instance, a range from about 100 to about 200 also includes ranges from 110 to 150, 170 to 190, 153 to 162, and 145.3 to 149.6. Further, a limit of up to about 7 also includes a limit of up to about 5, up to 3, and up to about 4.5, as well as ranges within the limit, such as from about 1 to about 5, and from about 3.2 to about 6.5.

The term “thermoplastic” is used herein to mean any material formed from a polymer which softens and flows when heated above certain polymer-dependent elevated temperatures and solidifies when cooled below that temperature; such a polymer may be heated and softened and cooled and solidified a number of times without suffering any basic alteration in characteristics, provided heating is below the decomposition temperature of the polymer. Examples of thermoplastic polymers include, by way of illustration only, polyolefins, polyesters, polyamides, polyurethanes, fluoropolymers, acrylic ester polymers and copolymers, polyvinyl chloride, polyvinyl acetate, etc. and copolymers thereof that are safe to use within a body of a patient.

As used herein, the terms “covered stent” or “vascular covered stent” mean an expandable, flexible, metal tube-shaped device (i.e., stent) that is covered with a fabric or graft material, such as a stretchable thermoplastic material, for example but not limited to, an expandable polytetrafluoroethylene (ePTFE) or a polyester that are safe to use within a body of a patient. Each covered stent is mounted on the end of a delivery catheter system.

As used herein, the term “tubular structure” means a hollow structure that has an inner surface and an outer surface which can have about the same inner diameter and about the same outer diameter or about the same width and about the same height across a length of the tubular structure, such as a cylindrical structure or can have an inner diameter and/or a outer diameter that is varied or widths and heights that are varied along a length of the tubular structure.

As used herein, the term “skeletal frame” means a supportive or protective structure that gives shape and strength to the stent on which stretchable thermoplastic interior cover of a stent can be secured. The skeletal frame may be porous and expandable.

As used herein, the terms “stretchable thermoplastic interior cover” means a fabric such as a nonwoven fabric, film or composite or laminate material that can be used to cover a can be safely used within a body of a patient.

As used herein, the terms “frustoconical shape” or “frustoconical” means a structure or a portion of the structure having the shape of a frustum of a cone or pyramid, for example, having a truncated cone shape or a truncated pyramidal shape.

As used herein, the term “vascular structure” means a vessel within a human body used to transport or circulate blood with the body. The vessel can be natural or artificial and can include, but not limited to human veins or arteries, artificial or synthetic veins or arteries, and dialysis access device.

The present subject matter relates to endovascular devices and endovascular device systems and related methods that allow for the modulation/regulation of vascular flow. In particular, the present subject matter relates to endovascular stents and endovascular device systems and related methods that allow for the modulation and/or regulation of vascular flow to treat certain disease states.

The present subject matter provides endovascular stents and endovascular device platforms. can comprise a balloon-stent system that is inserted using minimally invasive techniques. The balloon-stent system can comprise of a dumbbell, stepped or funnel shaped angioplasty balloon with a mounted covered, vascular stent. The angioplasty balloon is used for inserting, positioning and expanding the vascular stent in a desired blood vessel. Once inserted, the stent will modulate and even reduce vascular flow. Methods related to the manufacture and use of the endovascular stents and endovascular device platforms as disclosed herein are also provided.

A dumbbell or funnel shaped balloon with a mounted covered stent can be inserted into a vascular structure with the balloon being inflated to expand the stent and then deflated for removal leaving the stent within the vascular structure to alter vascular flow and specifically reduce blood flow. Poiseuille's formula states that flow can be reduced in a tube that carries a fluid by reducing the diameter (the Poiseuille's formula express the discharged streamlined volume flow through a smooth-walled circular pipe: v=πp r 4/8η|(1) v=discharge volume flow (m 3/s) p=pressure difference between the ends of the pipe (n/m 2, pa). In short, reduction in the radius of a pipe, i.e., a vascular structure like a dialysis access device, can lead to a reduction in flow within that device to treat disease states such as vascular access-induced steal syndrome (VASS). VASS can be an uncommon but challenging complication that occurs due to a functioning arteriovenous (AV) fistula or graft in 6% of chronic kidney disease patients who require hemodialysis. In the case of VASS, the expanded stent can treat patient ischemia. Importantly, the device could be reversed (if needed) with an additional endovascular procedure. The endovascular device platform can comprise a 2-part system that includes a balloon expandable endovascular covered stent, meeting diameter specifications specific to dialysis grafts and a dumbbell-shaped, stepped, or funnel-shaped delivery balloon that will expand both edges of the stent flush to the vessel wall, while leaving a tapered, funnel-shaped opening at the center (an hour-glass shape) to limit the blood flow through the stent, thus slowing the flow enough to alleviate the aforementioned ischemia caused by VASS. A function of the installed stent relies on redirecting some of the patient's vascular flow to the dialysis access device. Too much flow from the artery to the vein via the dialysis access device will lead to ischemia or steal. Once installed, the covered stent with the reduced flow shape can alter, modulate, or restrict blood flow to treat ischemia.

Other disease states that will likely benefit from endovascular devices and endovascular device systems and related methods discussed herein. Specifically, congestive heart failure and inherited or acquired arterio-venous fistula/malformations.

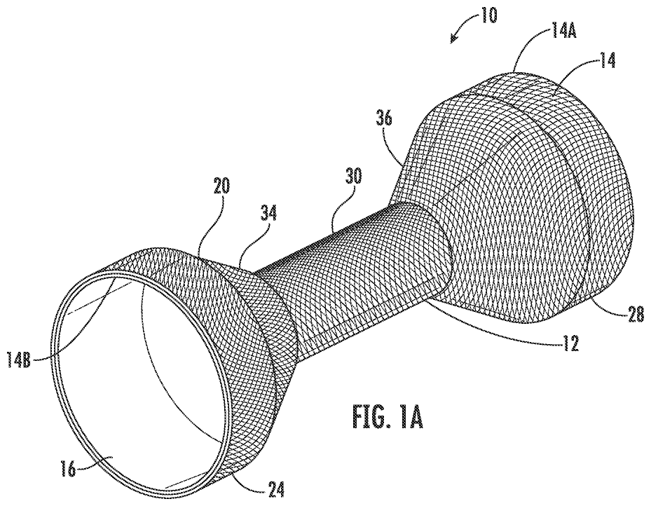

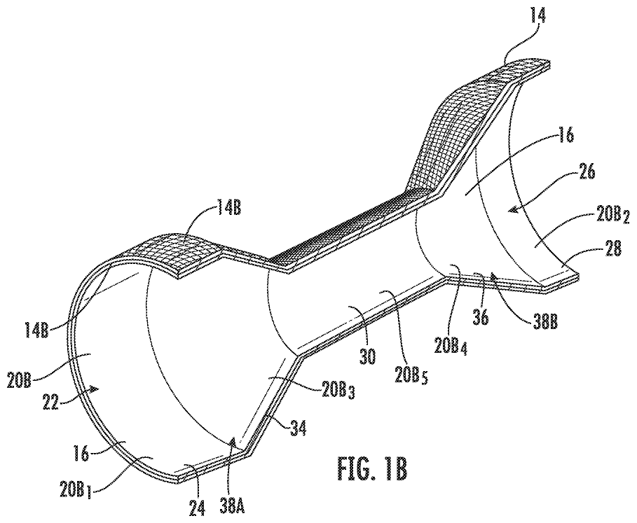

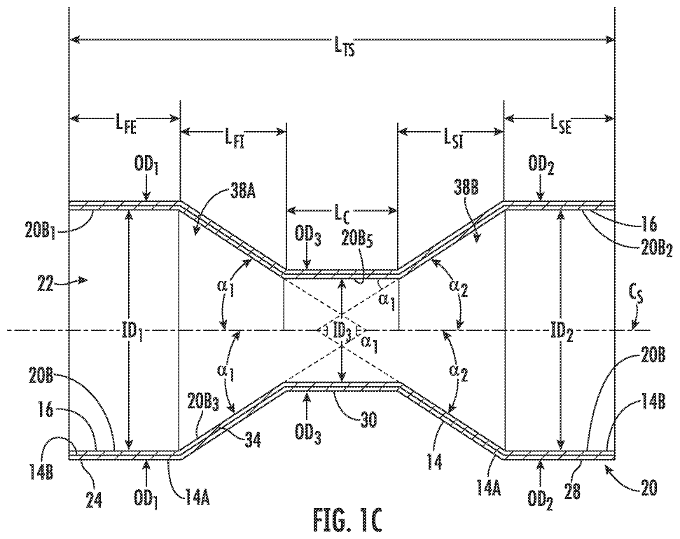

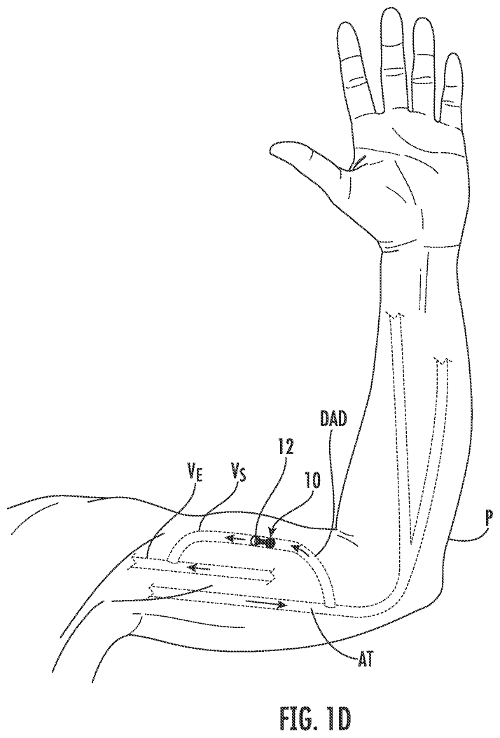

Referring to FIGS. 1A-1D, an embodiment of a covered stent, generally designated 10, in its installed shape 12 after deployment is illustrated. As shown in FIG. 1D, the covered stent 10 in the installed shape 12, can be deployed in a vascular structure VS in a patient P to reduce or restrict blood flow therethrough. Depending on the usage of the covered stent 10, the vascular structure VS can be a native vein VE or a native artery AT or can be an artificial vascular structure. For example, as shown in FIG. 1D, the vascular structure VS can be a dialysis access device DAD that is used for hemodialysis that can be connected to both a vein VE an an artery AT in an arm of the patient P. The covered stent 10 can be deployed and positioned in the dialysis access device DAD to restricted blood flow through a portion of the dialysis access device DAD to treat ischemia, for example. As shown in FIGS. 1A-1C, the covered stent 10 can comprise a dumbbell shaped tubular structure 20 having a first inner diameter ID₁ of a first end portion 24 and a second inner diameter ID₂ of a second end portion 28 that are larger than a third inner diameter ID₃ within a center portion 30 of the tubular structure 20.

For example, in some embodiments, the first inner diameter ID₁ of a first end portion 24 and a second inner diameter ID₂ of a second end portion 28 can be about 10% to about 70% larger in diameter than the third inner diameter ID₃ within a center portion 30 of the tubular structure 20. In some embodiments, the first inner diameter ID₁ of a first end portion 24 and a second inner diameter ID₂ of a second end portion 28 can be about 25% to about 60% larger in diameter than the third inner diameter ID₃ within a center portion 30 of the tubular structure 20. In some embodiments, the first inner diameter ID₁ of a first end portion 24 and a second inner diameter ID₂ of a second end portion 28 can be about 30% to about 55% larger in diameter than the third inner diameter ID₃ within a center portion 30 of the tubular structure 20. In some embodiments, the first inner diameter ID₁ of a first end portion 24 and a second inner diameter ID₂ of a second end portion 28 can be about 40% to about 50% larger in diameter than the third inner diameter ID₃ within a center portion 30 of the tubular structure 20. For instance, in some embodiments, the first inner diameter ID₁ of a first end portion 24 and a second inner diameter ID₂ of a second end portion 28 can be about 50% larger in diameter than the third inner diameter ID₃ within a center portion 30 of the tubular structure 20.

The tubular structure 20 can comprise a first intermediate portion 34 between the first end portion 24 and the center portion 30 and a second intermediate portion 36 between the second end portion 28 and the center portion 30. For example, the first and second intermediate portions 24, 28 each can have a truncated cone shape. For instance, the first and second intermediate portions 24, 28 each can have a frustoconical shape. In particular, the first intermediate portion 34 can gradually narrow from the first inner diameter ID₁ of the first end portion 24 to the third inner diameter ID₃ of the center portion 30. Similarly, the second intermediate portion 36 can gradually narrow from the second inner diameter ID₂ of the second end portion 28 to the third inner diameter ID₃ of the center portion 30 to facilitate laminar flow of blood passing through the stent 10 once the stent is installed.

In some embodiments, the tubular structure 20 of the covered stent 10 can comprise a skeletal frame 14 configured in a tubular shape and having an exterior 14A and an interior 14B. The skeletal frame 14 can comprise a metal. For example, in some embodiments, the skeletal frame can be braided metal wire. For example, in some embodiments, the skeletal frame 14 can comprise a stainless steel. In some embodiments, the skeletal frame 14 can comprise a wire mesh that can be expanded to the installed shape 12 of the covered stent 10. In some embodiments, the skeletal frame 14 can comprise a braid of metal wires.

Further, the tubular structure 20 of the covered stent 10 can also comprise a stretchable interior cover 16 configured to the interior 14B of the skeletal frame 14 that forms an interior surface 20B of the tubular structure 20. In particular, once the interior surface is expanded to its installed shape 12, the interior surface 20B can comprise different interior surface sections that can have the same surface structure but different orientations and positions within the covered stent 10. For example, the interior surface 20B can comprise a first interior surface section 20B₁ in the first end portion 24 and a second interior surface section 20B₂ in the second end portion 28 in the cover stent 10 that are circumferential in nature. Similarly, the interior surface 20B can comprise a sloping, or tapering, third interior surface section 20B₃ in the first intermediate portion 34 and a fourth sloping, or tapering, interior surface section 20B₄ in the second intermediate portion 36 in the cover stent 10, while having a smaller circumferential fifth interior surface section 20B₃ in the center portion 30 of the covered stent 10. Thus, the interior surface section 20B₁-20B₃ can have different profiles as shown in FIG. 1C.

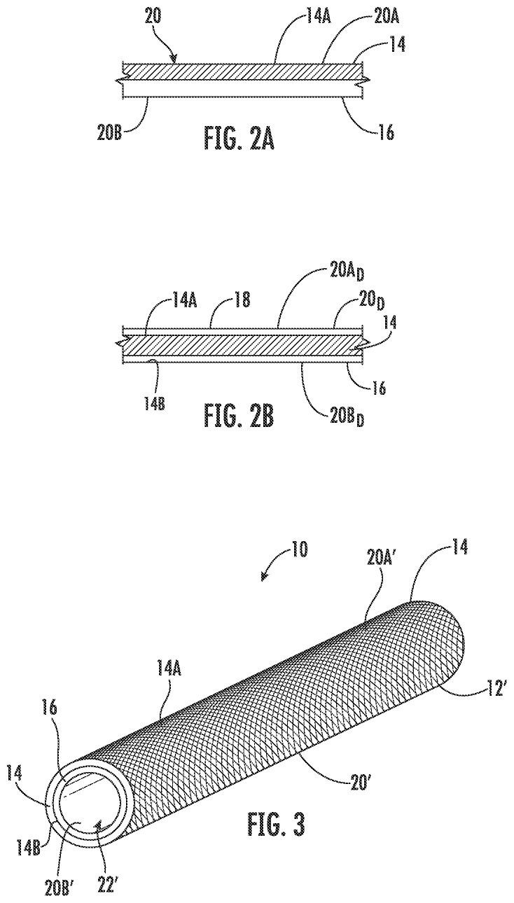

Portions of tubular structures are provided in FIGS. 2A and 2B. As shown in FIGS. 2A and 2B, the tubular structure can be formed in different manners. The portion of a tubular structure 20 shown in FIG. 2A is similar in construction to the tubular structure 20 shown in FIGS. 1A-1D. As shown in FIG. 2A, in some embodiments, the interior cover 16 can form the interior surface 20B and exterior 20A of the tubular structure 20 can be formed by the exterior 14A of the skeletal frame 14. A portion of a tubular structure 20_{D} is shown in FIG. 2B that has a different construction from the tubular structure shown in FIG. 2A. In some embodiments, as shown in FIG. 2B, the tubular structure 20_{D} of the covered stent 10 can comprise a stretchable exterior cover 18 configured to the exterior 14A of the skeletal frame 14 that can form an exterior surface 20A_{D} of the tubular structure 20_{D} with the interior cover 16 forming the interior surface 20B_{D}. The stretchable interior cover 16 and exterior cover 18 of the different embodiments can comprise a stretchable thermoplastic material that is safe to use within a body of a patient. For example, in some embodiments, the stretchable interior cover 16 and the stretchable exterior cover 18 can comprise a polytetrafluoroethylene (PTFE). In some embodiments, the stretchable interior cover 16 and the stretchable exterior cover 18 can comprise a polyester, such as polyester sold under the brand name DACRON, manufactured and sold by DuPont de Nemours. Inc, headquartered in Wilmington, Del.

In the installed shape 12, the different portions of the tubular structure 20 can have different shapes to provide different features to the function of the covered stent 10. For example, the first end portion 24 of the tubular structure 20 of the covered stent 10 can have a hollow cylindrical shape 22 that is defined by the first inner diameter ID₁ as measured from the interior surface section 20B₁ of the tubular structure 20 within the first end portion 24. Similarly, the second end portion 28 can have a hollow cylindrical shape 26 that is defined by the second inner diameter ID₂ as measured from the interior surface section 20B₂ of the tubular structure 20 within the second end portion 28. The first and second end portions 24, 28 can provide the exterior surface portions that abut against the walls of the vascular structure VS to provide a surface for grafting the cover stent 10 to the vascular structure VS and anchoring the cover stent 10

To restricts blood flow through the tubular structure 20 of the covered stent 10 in the vascular structure in which the stent 10 is placed, the third inner diameter ID₃ of the center portion 30 is less than the first inner diameter ID₁ and the second inner diameter 28. To facilitate laminar blood flow through the cover stent 10 as well as on the down flow side of the covered stent 10, the first and second intermediate portions 34, 36 each having a frustoconical shape 38A, 38B. For example, the interior surface 20B of the tubular structure 20 in the first intermediate portion 34 can taper from the interior surface 20B of tubular structure 20 at the first inner diameter ID₁ of the first end portion 24 to the interior surface 20B of the tubular structure 20 at the third inner diameter ID₃ of the center portion 30. Similarly, the interior surface 20B of the tubular structure 20 in the second intermediate portion 36 can taper from the interior surface 20B of tubular structure 20 at the second inner diameter ID₂ of the second end portion 28 to the interior surface 20B of the tubular structure 20 at the third inner diameter ID₃ of the center portion 30. These tapered walls of the first and second intermediate portions 34, 36 each can have an angle of slope α₁, α₂ of the interior surfaces 20B in the respective first and second intermediate portions 34, 36 as measured from a centerline axis CL of the tubular structure 20 that extends along the length L_{TS}. These angles of slope α₁, α₂ can be gradual enough in their steepness to facilitate laminar flow of blood passing through the covered stent 10.

In particular, to facilitate laminar flow of blood passing through the covered stent 10, the first intermediate portion 34 can gradually narrow from the first inner diameter ID₁ of the first end portion 24 to the third inner diameter ID₃ of the center portion 30. Similarly, the second intermediate portion 28 can gradually narrow from the second inner diameter ID₂ of the second end portion 28 to the third inner diameter ID₃ of the center portion 30 to facilitate laminar flow of blood passing through the covered stent 10. To provide for the gradual narrowing, the first and second intermediate portions can comprise a substantial portion of the length L_{TS} of the tubular structure 20. For example, in some embodiments, the first intermediate portion 34 and the second intermediate portion 36 of the tubular structure 20 each can comprise between about 15% of the length Ls of the tubular structure 20 and about 33% of the length L_{TS} of the tubular structure 20. For instance, in some embodiments, the first intermediate portion 34 and the second intermediate portion 36 of the tubular structure 20 each can comprise about 20% of the length of the tubular structure.

To also facilitate laminar flow of the blood through the covered stent 10, for example, in some embodiments, the center portion 30 can comprise between about 13% of the length L_{TS} of the tubular structure 20 and about 40% of the length L_{TS} of the tubular structure 20. In some embodiments, the center portion 30 of the tubular structure 20 can comprise about 30% of the length L_{TS} of the tubular structure 20.

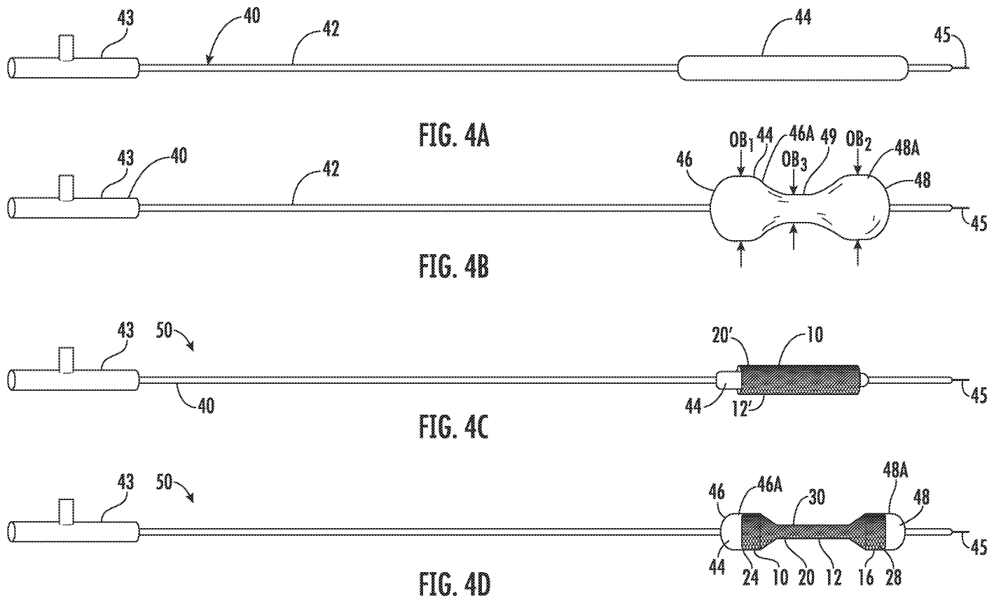

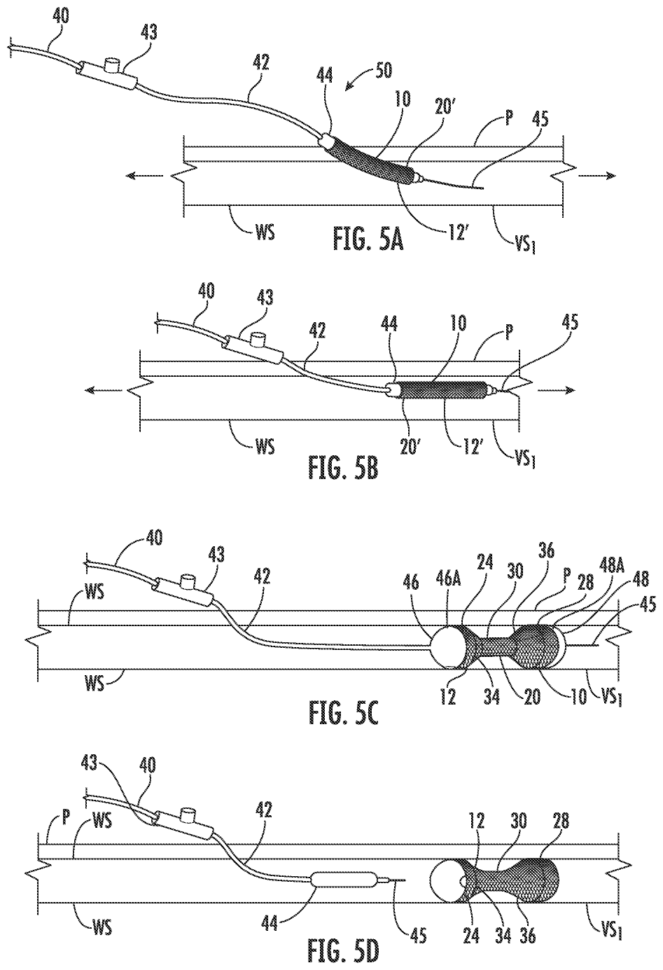

As stated above, the tubular structure 20 can be expandable. For example, the tubular structure 20 of the covered stent 10 can be expandable from a uniform tubular structure to the installed shape 12 using a catheter and balloon. More particularly, as shown in FIGS. 4A-4D and 5A-5D, the tubular structure 20 of the covered stent 10 can be expanded from a uniform tubular structure 20′ to the installed shape 12 of the covered stent 10 using a balloon catheter 40 that can comprise a shaft 42 and an angioplasty balloon 44 position on the shaft 42. For example, an endovascular stent-balloon system, generally designated 50, can be provided for installing the stent 10 within a vascular structure VS, such as a vein or artery of a patient or dialysis access device, for example. The endovascular stent-balloon system 50 can be configured to install the fluid flow restricting stent 10 in a vascular structure using a special balloon catheter 40 as shown in FIGS. 4A and 4B. The balloon catheter 40 can comprise a stem, or a shaft, 42 and an angioplasty balloon 44 on an end of the stem 44. In FIGS. 4A and 4C, the angioplasty balloon 44 is in an uninflated state. In the uninflated state, the profile of the angioplasty balloon 44 can be smaller and closer to that of the stem 42 to allow for easier percutaneous insertion into and transluminal passage of the endovascular stent-balloon system 50 through the vascular structure VS (see FIGS. 5A-5D). Once the catheter 40 of the endovascular stent-balloon system 50 has positioned the stent 10 in a desired position within the vascular structure VS, the angioplasty balloon 44 can be inflated. When Inflated as shown in FIG. 4B, the angioplasty balloon 44 can have a dumbbell shape with a first head 46A on a first end 46 of the angioplasty balloon 44 and a second head 48A on a second end 48 of the angioplasty balloon 44. The first head 46A on the first end 46 of the angioplasty balloon 44 can have a first outer diameter OB₁, while the second head 48A on the second end 48 of the angioplasty balloon 44 can have a second outer diameter OB₂. Between the first head 46A and the second head 48A of the inflated angioplasty balloon 44, the angioplasty balloon 44 can have a narrow center portion 49 as compared to the first head 46A and the second head 48A. The narrow center portion 49 can have a third outer diameter OB₃ that is less than the first outer diameter OB₁ of the first head 46A and the second outer diameter OB₂ of the second head 48A. The shape of the first and second heads 46A, 48A of the inflated balloon 44 can be such that the first and second heads 46A, 48A slope from the portion having the first outer diameter OB₁ and the second outer diameter OB₂, respectively, toward the narrow center portion 49 having the third outer diameter OB₃.

The covered stent 10 can be installed in a variety of different vascular structures VS. For example, the vascular structure VS can comprise at least one of a vein or artery with the patient. Additionally, the vascular structure VS can comprise a synthetic vascular structure not native to the patient. For example, the vascular structure VS can comprise a dialysis access device for use in hemodialysis.

As shown in FIG. 4C, an expandable covered stent 10′ can be mounted on the uninflated angioplasty balloon 44. expandable covered stent 10′ can comprise an unexpanded tubular structure 20′ having a uniform hollow cylindrical shape. As shown in FIG. 4D, upon the inflation of the angioplasty balloon 44, the covered stent 10 is expanded into an installed dumbbell shape 12 having the first inner diameter ID₁ of the first end portion 24 of the expanded tubular structure 20 and a second inner diameter ID₂ of a second end portion 28 of the tubular structure 20 that are larger than a third inner diameter ID₃ within a center portion 30 of the tubular structure 20 as shown in FIGS. 1A-2B. Once the expanded stent 10 is installed in the desired position in the vascular structure VS, the angioplasty balloon 44 can be deflated and the balloon catheter 40 can be removed from the vascular structure VS. In some embodiments, the first outer diameter OB₁ of the first head 46A of the balloon 44 and the second outer diameter OB₂ of the second head portion 48A of the balloon 44 can be substantially equal. So, the first inner diameter ID₁ of the first end portion 24 of the tubular structure 20 and the second inner diameter ID₂ of the second end portion 28 of the tubular structure 20 can be substantially equal.

As described above, upon inflation of the angioplasty balloon 44 and installation of the covered stent 10, the tubular structure 20 comprises a first intermediate portion 34 between the first end portion 24 and the center portion 30 and a second intermediate portion 36 between the second end portion 28 and the center portion 30 with the first and second intermediate portions 34, 36 each having a frustoconical shape. In particular, the first intermediate portion 34 can gradually narrow from the first inner diameter ID₁ of the first end portion 24 to the third inner diameter ID₃ of the center portion 30 and the second intermediate portion 36 can gradually narrow from the second inner diameter ID₂ of the second end portion 28 to the third inner diameter ID₃ of the center portion 30 on the other side of the stent 10 to facilitate laminar flow of blood passing through the stent 10.

The expanded stent 10 can be helpful in treating ischemia within a dialysis patient. The vascular structure VS can comprise a dialysis access device. The expanded tubular structure 20 of the covered stent 10 can also have a first outer diameter OD₁ of the first end portion 24 and a second outer diameter OD₂ of the second end portion 28. Upon installation within the vascular structure VS of the dialysis access device, the first outer diameter OD₁ of the first end portion 24 of the tubular structure 20 and the second outer diameter OD₂ of the second end portion 28 of the tubular structure 20 meet diameter specifications specific to dialysis grafts so that the covered stent 10 will graft to the vascular structure VS of the dialysis access device to hold the covered stent 10 in place. Once in place in the vascular structure VS of the dialysis access device, the third inner diameter ID₃ of center portion 30 is such that the third inner diameter ID₃ reduces flow of blood within the dialysis access device to treat ischemia within the dialysis patient.

As described above, the unexpanded tubular structure 20′ that has a uniform hollow cylindrical shape can comprise the skeletal frame 14 configured in a uniform tubular shape and having the exterior 14A and the interior 14B and a stretchable thermoplastic interior cover 16 configured to the interior 14B of the skeletal frame 14 that forms an interior surface 20B′ of the tubular structure 20′. The skeletal frame 14 and the internal cover 16 are expandable. In some embodiments, the skeletal structure 14 can comprise a metal such as a wire braid for example. In some embodiments, the stretchable thermoplastic interior cover 16 can comprise polytetrafluoroethylene. In some embodiments, the stretchable interior cover 16 can comprise a polyester.

Once the angioplasty balloon 44 has been inflated and expanded the tubular structure 20 so that he the covered stent 10 is in the installed shape 12, the first end portion 24 of tubular structure 20 has a hollow cylindrical shape with the first inner diameter ID₁ as measured from the interior surface 14B of the tubular structure 20 generally uniformly throughout the first end portion 24. Similarly, the second end portion 28 can have a hollow cylindrical shape with the second inner diameter ID₂ as measured from the interior surface 14B of the tubular structure 20 throughout the second end portion 28. As outlined above, the center portion 30, which can also have a hollow cylindrical shape, along the length between the first end portion 24 and the second end portion 28 with the third inner diameter ID₃ at the center portion 30 being less the first inner diameter ID₁ and the second inner diameter ID₂ such that the third inner diameter ID₃ at the center portion 30 restricts blood flow in the dialysis access device in which the stent 10 is placed. As above, when the balloon 44 is inflated, the shape of the balloon 44 with the inward sloping, or tapering, first and second heads 46A, 48A also expands the tubular structure to have the first intermediate portion 34 between the first end portion 24 and the center portion 30 and a second intermediate portion 36 between the second end portion 28 and the center portion 30. The first and second intermediate portions 36 and 38 can each have a frustoconical shape with the interior surface 20B of the tubular structure 20 in the first intermediate portion 34 tapering from the interior surface 20B of tubular structure 20 at the first inner diameter ID₁ of the first end portion 24 to the interior surface 20B of the tubular structure 20 at the third inner diameter ID₃ of the center portion 30 and the interior surface 208 of the tubular structure 20 in the second intermediate portion 36 tapering from the interior surface 20B of tubular structure 20 at the second inner diameter ID₂ of the second end portion 28 to the interior surface 20B of the tubular structure 20 at the third inner diameter ID₃ of the center portion 30.

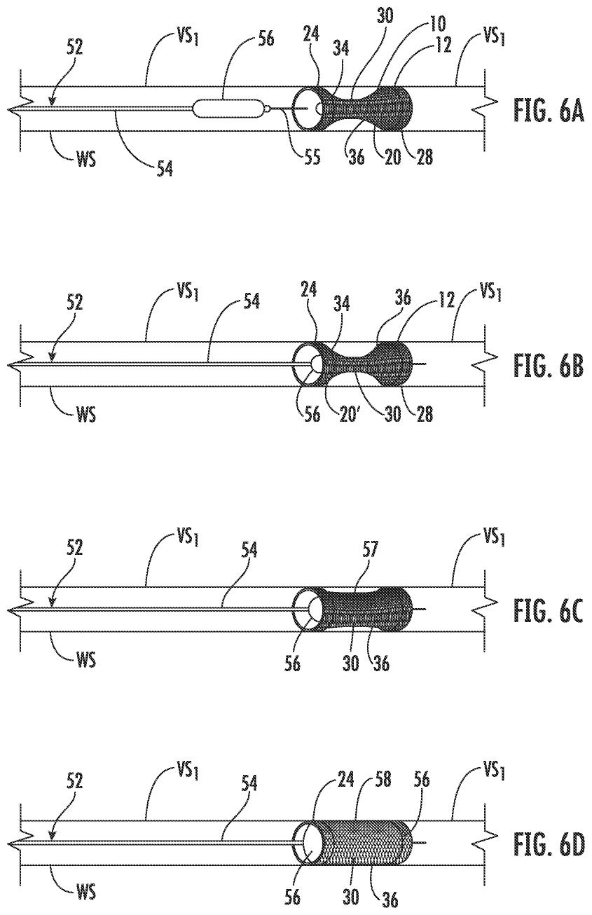

Thus, as shown in FIGS. 6A-6D, a method for restricting blood flow restricting within a dialysis access device VS₁ to treat ischemia within a dialysis patient using an endovascular stent-balloon system is provided. The stent and the procedure for installing can be used to aid in reducing or preventing blood from flowing back into the vein into which the dialysis access device VS₁ is connected. As shown in FIG. 6A, a stent-balloon catheter 50 can be provided that can comprise a catheter 40 and an expandable covered stent 10 on the catheter 40. The catheter 40 can comprise a shaft 42 and an angioplasty balloon 44 on a portion of the shaft 42. The expandable covered stent 10 can comprise an unexpanded tubular structure 20′ having a uniform hollow cylindrical shape that is mounted on the uninflated angioplasty balloon 44.

Once the dialysis access device VS is located and the patient is prepared for the installation of the stent, the stent-balloon catheter 50 can be inserted into the body of the patient at an entry point. Depending on the location of the dialysis access device VS₁, the stent-balloon catheter 50 can be inserted into a dialysis access device VS₁ within a patient through the arm for example. In other embodiments of the method, the stent-balloon catheter 50 can be inserted at another entry point and maneuvered through the circulatory system to the dialysis access device VS₁. Once in the dialysis access device VS₁, the stent-balloon catheter 50 is positioned in the dialysis access device VS₁ within a patient in a position where restriction of blood flow is desired.

The angioplasty balloon 44 of the stent-balloon catheter 50 is then inflated to expand the unexpanded tubular structure 20′ of the covered stent 10. The angioplasty balloon 44 can have a dumbbell shape when inflated with a first head 46A on a first end 46 of the angioplasty balloon 44 having a first outer diameter OB₁ and a second head 48A on a second end 48 of the angioplasty balloon 44 having a second outer diameter OB₂ and a narrow center portion 49 having a third outer diameter OB₃ that is less than the first outer diameter OB₁ of the first end 46 and the second outer diameter OB₂ of the second end 48. Thus, when the angioplasty balloon 44 is inflated, the covered stent 10 is expanded into an installed dumbbell shape 12 having a first inner diameter ID₁ of a first end portion 24 of the tubular structure 20 and a second inner diameter ID₂ of a second end portion 28 of the tubular structure 28 that are larger than a third inner diameter ID₃ within a center portion 30 of the tubular structure 20 with the first and second end portions 24, 28 being flush against wall segments WS of the dialysis access device VS.

The angioplasty balloon 44 of the balloon catheter 40 can be deflated and the catheter 40 can be removed from the installed covered stent 10 and the dialysis access device VS₁ leaving the covered stent 10 in place in a position to restrict blood flow within the dialysis access device VS₁ to treat ischemia within the patient.

Once installed, the covered stent 10 has the benefit of being able to be expanded to permit increased blood flow if complication due to the restricted blood flow arises. For example, the covered stent 10 can be installed as described above with the catheter 40 removed and the exterior surface 14A of the first and second end portions 24, 28 of the covered stent 10 are grafted into the vascular structure, such as the dialysis access device VS₁. If the third inner diameter ID₃ of the center portion 30 of covered stent 10 causes a restriction in the blood flow that interferes with the hemodialysis process or causes circulatory problems or pain in the patient, then the third inner diameter ID₃ of the center portion 30 of covered stent 10 can be expandable using a different expansion catheter 52 from the catheter 40 used to install the covered stent 10 in its installed shape 12 as shown in FIGS. 6A-6D. Thus, the tubular structure 20 can be expandable such that the third inner diameter ID₃ within the center portion 30 is capable of being expanded after installation using a balloon catheter. In this manner, the covered stent 10 can be expanded to an emergency flow shape. For example, after installation, the tubular structure 20 can be configured to be uniformly expandable with a balloon catheter such that the installed shape is changed such that the first, second and third inner diameters ID₁, ID₂, ID₃ are substantially equal and the interior surface 20B of the tubular structure 20 has a generally uniform cylindrical shape that can be flush with the walls of the vascular structure.

In some embodiments of a method of expanding the third inner diameter ID₃ of the center portion 30 of covered stent 10, the expansion catheter 52 can be inserted into the vascular structure, such as the dialysis access device VS₁ shown in FIG. 6A. the expansion catheter 52 can comprise a stem, or shaft 54 and an angioplasty balloon 56 that can be inflated and deflated as needed. In particular, the expansion catheter 52 can be inserted into the dialysis access device VS₁ and the third inner diameter ID₃ of the center portion 30 of tubular structure 20 of covered stent 10 with the angioplasty balloon 56 uninflated as shown in FIG. 6B. Once the expansion catheter 52 is in a desired position in the covered stent 10 with at least a portion in the third inner diameter ID₀ of the center portion 30, the angioplasty balloon 56 can be inflated to uniformly expand the third inner diameter ID₃ of the center portion 30 of the tubular structure 20 of the covered stent 10 such that the third inner diameter ID₃ within the center portion 30 is expanded to increase blood flow in the dialysis access device VS₁ at the position of the covered stent 10. As shown in FIG. 6C, angioplasty balloon 56 can be partially inflated so that the third inner diameter ID₃ within the center portion 30 can be expanded in a manner that it still restricts the blood flow but to a lesser extent than the installed shape 12 of the covered stent 10 as shown in FIG. 6A. Additionally, the angioplasty balloon 56 can be fully inflated to fully expand the third inner diameter ID₃ of the center portion 30 of the tubular structure 20 of the covered stent 10 such that the first, second and third inner diameters ID₁, ID₂, ID₃ can be substantially equal and the interior surface 20B of the tubular structure 20 has a uniform cylindrical shape 58 to increase blood flow in the dialysis access device VS₁ at the position of the covered stent 10. In this manner, the third inner diameter ID₃ of the center portion 30 of the tubular structure 20 of the covered stent 10 can be expanded or widened to be equal to the first and second inner diameters ID₁, ID₂ such that the tubular structure 20 is flush with the vessel wall.

Thus, as disclosed herein, a method for restricting blood flow within a vascular structure using an endovascular stent-balloon system is provided. A stent-balloon catheter can be provided and can comprise a shaft and an angioplasty balloon on a portion of the shaft and an expandable covered stent comprising an unexpanded tubular structure that is mounted on the uninflated angioplasty balloon. The angioplasty balloon can have a dumbbell shape when it is inflated with a first head on a first end of the angioplasty balloon having a first outer diameter and a second head on a second end of the angioplasty balloon having a second outer diameter and a narrow center portion having a third outer diameter that is less than the first outer diameter of the first end and the second outer diameter of the second end.

The stent-balloon catheter can be positioned in a vascular structure within a patient in a position where restriction of blood flaw is desired. The angioplasty balloon of the stent-balloon catheter can be inflated such that the covered stent is expanded into an installed dumbbell shape having a first inner diameter of a first end portion of the tubular structure and a second inner diameter of a second end portion of the tubular structure that are larger than a third inner diameter within a center portion of the tubular structure between the first and second end portions. The stent and the balloon catheter can be sized and/or selected to fit within the vascular structure of the patient so that, when the angioplasty balloon is inflated the stent secured in position within the vascular structure. Once the stent is expanded and secured within the vascular structure, the angioplasty balloon of the stent-balloon catheter can be deflated. The stent-balloon catheter can then be removed from the vascular structure leaving the covered stent in place in a position to restrict blood flow within the vascular structure within the patient.

These and other modifications and variations to the present subject matter may be practiced by those of ordinary skill in the art, without departing from the spirit and scope of the present subject matter, which is more particularly set forth herein above and any appending claims. In addition, it should be understood the aspects of the various embodiments may be interchanged either in whole or in part. Furthermore, those of ordinary skill in the art will appreciate that the foregoing description is by way of example only, and is not intended to limit the present subject matter.

1. A covered stent that, upon deployment, is configured to an installed shape, the covered stent configured in the installed shape comprising a dumbbell shaped tubular structure having a first inner diameter of a first end portion and a second inner diameter of a second end portion that are larger than a third inner diameter within a center portion of the tubular structure.

2. The covered stent according to claim 1, wherein the tubular structure comprises a first intermediate portion between the first end portion and the center portion and a second intermediate portion between the second end portion and the center portion, the first and second intermediate portions each having a frustoconical shape.

3. The covered stent according to claim 2, wherein the first intermediate portion narrows from the first inner diameter of the first end portion to the third inner diameter of the center portion and the second intermediate portion narrows from the second inner diameter of the second end portion to the third inner diameter of the center portion at a slope that facilitates laminar flow of blood passing through the stent.

4. The covered stent according to claim 1, wherein the tubular structure is expandable from a uniform tubular structure to the installed shape using a balloon catheter comprising a shaft and an angioplasty balloon position on the shaft.

5. The covered stent according to claim 4, wherein, after installation, the tubular structure is configured to be uniformly expandable with a balloon catheter such that the installed shape is changed such that the first, second and third inner diameters are substantially equal and the interior surface of the tubular structure has a generally uniform cylindrical shape.

6. The covered stent according to claim 4, wherein the tubular structure is expandable such that the third inner diameter within the center portion is capable of being expanded after installation using a balloon catheter.

7. The covered stent according to claim 1, wherein the first end portion of the tubular structure comprises a first outer diameter and the second end portion of the tubular structure comprises a second outer diameter and, for installation within a dialysis access device, the first outer diameter of the first end portion of the tubular structure and the second outer diameter of the second end portion of the tubular structure meet diameter specifications specific to dialysis grafts.

8-11. (canceled)

12. The covered stent according to claim 3, wherein the first intermediate portion and the second intermediate portion of the tubular structure each comprises between about 0.15% of the length of the tubular structure and about 0.33% of the length of the tubular structure.

13-28. (canceled)

29. An endovascular stent-balloon catheter system configured to install a fluid flow restricting stent in a vascular structure, the endovascular stent-balloon system comprising:
a balloon catheter comprising a stem and an angioplasty balloon on an end of the stem, the angioplasty balloon having a dumbbell shape when inflated with a first head on a first end of the angioplasty balloon having a first outer diameter and a second head on a second end of the angioplasty balloon having a second outer diameter and a narrow center portion having a third outer diameter that is less than the first outer diameter of the first end and the second outer diameter of the second end; and
an expandable covered stent comprising an unexpanded tubular structure having a uniform hollow cylindrical shape that is mounted on the uninflated angioplasty balloon;
upon insertion of the balloon catheter into a vascular structure in an appropriate position and the inflation of the angioplasty balloon, the covered stent is expanded into an installed dumbbell shape having a first inner diameter of a first end portion of the tubular structure and a second inner diameter of a second end portion of the tubular structure that are larger than a third inner diameter within a center portion of the tubular structure.

30. The endovascular stent-balloon catheter system according to claim 29, wherein, upon inflation of the angioplasty balloon and installation of the covered stent, the tubular structure comprises a first intermediate portion between the first end portion and the center portion and a second intermediate portion between the second end portion and the center portion, the first and second intermediate portions each having a frustoconical shape.

31. The endovascular stent-balloon catheter system according to claim 30, wherein the first intermediate portion gradually narrows from the first inner diameter of the first end portion to the third inner diameter of the center portion and the second intermediate portion gradually narrows from the second inner diameter of the second end portion to the third inner diameter of the center portion to facilitate laminar flow of blood passing through the stent.

32. The endovascular stent-balloon catheter system according to claim 30, wherein, after installation, the tubular structure is configured to be uniformly expandable with a balloon catheter such that the first, second and third inner diameters are substantially equal and an interior surface of the tubular structure having a generally uniform cylindrical shape.

33. The endovascular stent-balloon system according to claim 30, wherein the tubular structure is expandable such that the third inner diameter within the center portion is capable of being expanded after installation using a balloon catheter.

34. The endovascular stent-balloon catheter system according to claim 29, wherein the first end portion of the tubular structure comprises a first outer diameter and the second end portion of the tubular structure comprises a second outer diameter and, for installation within a dialysis access device, the first outer diameter of the first end portion of the tubular structure and the second outer diameter of the second end portion of the tubular structure meet diameter specifications specific to dialysis grafts

35. The endovascular stent-balloon catheter system according to claim 29, wherein the vascular structure comprises a dialysis access device and, upon installation within the dialysis access device, the third inner diameter of center portion is such that the third inner diameter reduces flow of blood to treat ischemia within a dialysis patient.

36-43. (canceled)

44. A method for restricting blood flow restricting within a dialysis access device to treat ischemia within a dialysis patient using an endovascular stent-balloon system, the method comprising:
providing a stent-balloon catheter comprising a shaft and an angioplasty balloon on a portion of the shaft and an expandable covered stent comprising an unexpanded tubular structure having a uniform hollow cylindrical shape that is mounted on the uninflated angioplasty balloon, the angioplasty balloon having a dumbbell shape when inflated with a first head on a first end of the angioplasty balloon having a first outer diameter and a second head on a second end of the angioplasty balloon having a second outer diameter and a narrow center portion having a third outer diameter that is less than the first outer diameter of the first end and the second outer diameter of the second end;
inserting the stent-balloon catheter into a dialysis access device within a patient;
positioning the stent-balloon catheter in the dialysis access device within a patient in a position where restriction of blood flow is desired;
inflating the angioplasty balloon of the stent-balloon catheter such that the covered stent is expanded into an installed dumbbell shape having a first inner diameter of a first end portion of the tubular structure and a second inner diameter of a second end portion of the tubular structure that are larger than a third inner diameter within a center portion of the tubular structure with the first and second end portions being flush against wall segments of the dialysis access device;
deflating the angioplasty balloon of the stent-balloon catheter; and
removing the stent-balloon catheter from the covered stent and the dialysis access device leaving the covered stent in place in a position to restrict blood flow within the dialysis access device to treat ischemia within the patient.

45. The method according to claim 44, further comprising inserting a second uninflated angioplasty balloon on a balloon catheter into the third inner diameter of the center portion of the tubular structure of the covered stent.

46. The method according to claim 45, further comprising inflating the second angioplasty balloon and uniformly expanding the third inner diameter of the center portion of the tubular structure of the covered stent such that the first, second and third inner diameters are substantially equal and an interior surface of the tubular structure has a uniform cylindrical shape to increase blood flow in the dialysis access device at the position of the covered stent.

47. The method according to claim 45, further comprising inflating the second angioplasty balloon and uniformly expanding the third inner diameter of the center portion of the tubular structure of the covered stent such that such that the third inner diameter within the center portion is expanded to increase blood flow in the dialysis access device at the position of the covered stent.

48. The method according to claim 45, wherein the first end portion of the tubular structure comprises a first outer diameter and the second end portion of the tubular structure comprises a second outer diameter and, for installation within a dialysis access device, the first outer diameter of the first end portion of the tubular structure and the second outer diameter of the second end portion of the tubular structure meet diameter specifications specific to dialysis grafts

49-55. (canceled)
